(19) United States
(12) Patent Application Publication
(10) Pub. No.: US 2015/0099943 A1
(43) Pub. Date: Apr. 9, 2015

(54) WEARABLE PHYSIOLOGICAL SENSING DEVICE WITH OPTICAL PATHWAYS

(71) Applicant: COVIDIEN LP, Mansfield, MA (US)

(72) Inventor: BRIAN KEITH RUSSELL, Annapolis, MD (US)

(21) Appl. No.: 14/504,577

(22) Filed: Oct. 2, 2014

Related U.S. Application Data
(60) Provisional application No. 61/887,031, filed on Oct. 4, 2013.
(60) Provisional application No. 61/891,644, filed on Oct. 16, 2013.

(51) Int. Cl. A61B 5/00; A61B 5/1455; A61B 5/0402; A61B 5/0488; A61B 5/11; A61B 5/0205; A61B 5/026

(52) U.S. Cl. CPC A61B 5/0059; A61B 5/02055; A61B 5/14551; A61B 5/0261; A61B 5/0488; A61B 5/11; A61B 5/0402; A61B 5/6802; A61B 5/7221; A61B 5/7278; A61B 5/7203; A61B 5/024
USPC 600/301; 600/476; 600/477; 600/323; 600/479; 600/546; 600/547; 600/587

(57) ABSTRACT

A wearable physiological sensing device with optical pathways is described. The wearable physiological sensing device may include at least one light source; a first light pipe coupled with the at least one light source, the first light pipe at least partially circumscribing an extremity of a patient, and including at least one aperture for radiating light from the light source into the extremity. The wearable physiological sensing device may also include a second light pipe including at least one aperture for receiving the light radiated through the extremity, the second light pipe at least partially circumscribing the extremity of the patient, and an optical receiver coupled with the second light pipe configured to receive the light and output one or more signals representative of the received light.

### CROSS-REFERENCE TO RELATED APPLICATIONS

This application claims priority to U.S. Provisional Patent Application No. 61/887,031, filed on Oct. 4, 2013, and U.S. Provisional Patent Application No. 61/891,644, filed on Oct. 16, 2013, the entireties of which are incorporated by reference herein.

### TECHNICAL FIELD

The present disclosure relates generally to physiological monitoring systems, and more particularly to a wearable physiological sensing device with optical pathways.

### BACKGROUND

Existing physiological optical sensing devices typically include pulse oximeters, which pass a beam of light through a patient's fingertip or earlobe in order to measure light absorption or reflection spectroscopy. Such known optical sensing devices are often not designed for routine use, and may be obtrusive, awkward, or uncomfortable to wear, particularly those sensing devices which require wired connections to a processing device. Other known physiological optical sensing devices include watch-style devices, which may be suitable for daily use but which may pass a beam of light through the posterior of the wrist where vasculature is obstructed by muscles, tendons, and bones, thereby limiting blood flow sensing capabilities. Additionally, these watch-style optical devices may only be suitable to detect capillary blood flow along the posterior of the wrist, and may be unable to detect blood flow through, for example, radial or ulnar veins or arteries, which are disposed on the anterior side of the wrist and which are unobstructed by the bones and musculature of the wrist.

Accordingly, practitioners and patients may benefit from a wearable physiological sensing device that is more versatile than existing devices in the type, accuracy and completeness of data that may be collected from a patient.

### SUMMARY

The described features generally relate to a wearable physiological sensing device, as well as methods for using the same. The wearable physiological sensing device may include at least one light source and a first light pipe coupled with the at least one light source. The first light pipe may at least partially circumscribe an extremity of a patient, and may include at least one aperture for radiating light from the at least one light source into the extremity of the patient. The sensing device may further include a second light pipe comprising at least one aperture for receiving the light radiated through the extremity. The second light pipe may also at least partially circumscribe the extremity of the patient. The sensing device may further include an optical receiver coupled with the second light pipe and configured to receive the light radiated through the extremity and output one or more signals representative of the received light. Additionally, the sensing device may include a processor configured to determine one or more physiological parameters of the patient based, at least in part, on the outputted one or more signals.

The first light pipe of the wearable physiological sensing device may, in some embodiments, include a plurality of light pipes, such that light radiated from the light source may travel down one or more of the light pipes. In some embodiments, the first light pipe may include multiple apertures spaced along a length of the first light pipe, and the second light pipe may include multiple apertures spaced along a length of the second light pipe. In some embodiments, the wearable physiological sensing device may include a plurality of internal reflectors positioned in the first light pipe and/or the second light pipe. In this way, light from the light source may be reflected off one or more of the plurality of internal reflectors and may be radiated through the one or more first light pipes into the extremity of the patient at one or more aperture locations, and may be received into the second light pipe at one or more aperture locations and reflected off one or more of the plurality of internal reflectors to travel to the optical receiver such that more accurate and complete physiological data may be determined.

By providing a plurality of apertures spaced along the length of each of the first light pipe and second light pipe, more reliable optical coupling may be achieved, and thus more accurate patient physiological parameters may be monitored. This is at least because the light radiated from the light source may enter the extremity of the patient at any one of the plurality of apertures positioned along the first light pipe and spaced around the circumference of the patient's extremity, such that at least a portion of the radiated light is likely to reach at least one of the plurality of apertures positioned along the second light pipe and be received at the optical receiver.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label with a dash and a second label that distinguishes among the similar components. If only the first reference label is used in the specification, the description is applicable to any one of the similar components having the same first reference label irrespective of the second reference label.

FIG. 1 is a system diagram of an example of a physiological sensing system;

FIG. 2 is a diagram of an example of a wearable physiological sensing device viewed circumscribing a cross-section of an extremity of a patient in accordance with various embodiments;

FIG. 3A is a diagram of an example of a first light pipe in a wearable physiological sensing device in accordance with various embodiments;

FIG. 3B is a diagram of an example of a second light pipe in a wearable physiological sensing device in accordance with various embodiments;

FIG. 4 is a block diagram of an example of a sensor apparatus in accordance with various embodiments;

FIG. 5 is a block diagram of an example of an optical sensor module in accordance with various embodiments;

FIG. 6 is a block diagram of an example of a sensor apparatus in accordance with various embodiments; and

FIGS. 7 and 8 are flowcharts of methods for physiological sensing of a patient, in accordance with various embodiments.

### DETAILED DESCRIPTION

Optical sensing devices that are typically worn around the wrist often include a single light source and a single optical sensor, each located near the posterior of the patient's wrist such that light radiated from the light source is passed only through the posterior of the patient's wrist in order to detect patient physiological parameters, such as blood oxygen saturation, blood flow, pulse, etc. Limiting the light radiation to the posterior of the patient's wrist, however, may limit the blood flow sensing capabilities of the sensing device, at least because blood flow through, for example, the radial or ulnar veins or arteries may be detected only at the anterior of the wrist. In addition, typical wrist-worn optical sensing devices may be uncomfortably tight to ensure constant contact between the light source and the patient's wrist. Should typical wrist-worn optical sensing devices be worn too lose, or inevitably shift during wear, proper optical coupling may not be achieved.

Additionally, because typical optical sensing devices radiate light through the patient's wrist at only a single point, and capture the radiated light at a single point, the accuracy and reliability of the readings are further compromised. Inaccurate or irregular blood flow readings may further be received due to “noise” in the wrist or extremity in the form of bones, muscles, and other physiological barriers. For example, light exiting the single point of radiation on the posterior of the patient's wrist may come into contact with a bone or tendon and may scatter, such that the light may not reach the single optical sensor, or may provide inaccurate light absorption or reflection spectroscopy data when received by the optical receiver due to the scattering effect. Thus, in typical optical sensing devices, the chances that a single light source will emit light that will radiate through the patient's wrist or other extremity, will come into contact with vasculature in the patient's extremity without being intercepted by other matter in the patient's extremity, and will optically couple with the single optical sensor such that accurate physiological measurements may be collected, are slim, particularly when the patient inevitably moves his wrist or other extremity such that the paths of light radiation are interrupted.

Some known optical sensing devices have attempted to overcome these issues by providing a plurality of light sources and/or optical sensors positioned around the circumference of the patient's extremity, such that radiated light has a better chance of optically coupling with the plurality of optical sensors. Providing multiple light sources and sensors, however, may make the sensing device bulky, or may increase costs of manufacturing or maintenance, and may require more frequent charging or refurbishing of the device. Thus, it may be beneficial to provide a wearable physiological sensing device having, for example, a light source and an optical receiver located near the posterior of the wrist or other extremity of the patient, and further including first and second light pipes at least partially circumscribing the extremity of the patient, where the light pipes may include one or more apertures positioned along the length of each of the first and second light pipes. In this way, light may be radiated into the extremity and received from the extremity at a plurality of points along the circumference of the patient's extremity. Accuracy and reliability of the physiological measurements may thus be achieved by increasing the likelihood of optical coupling even with patient or device movement or physiological “noise” in the extremity, without increasing the bulk of the wearable device.

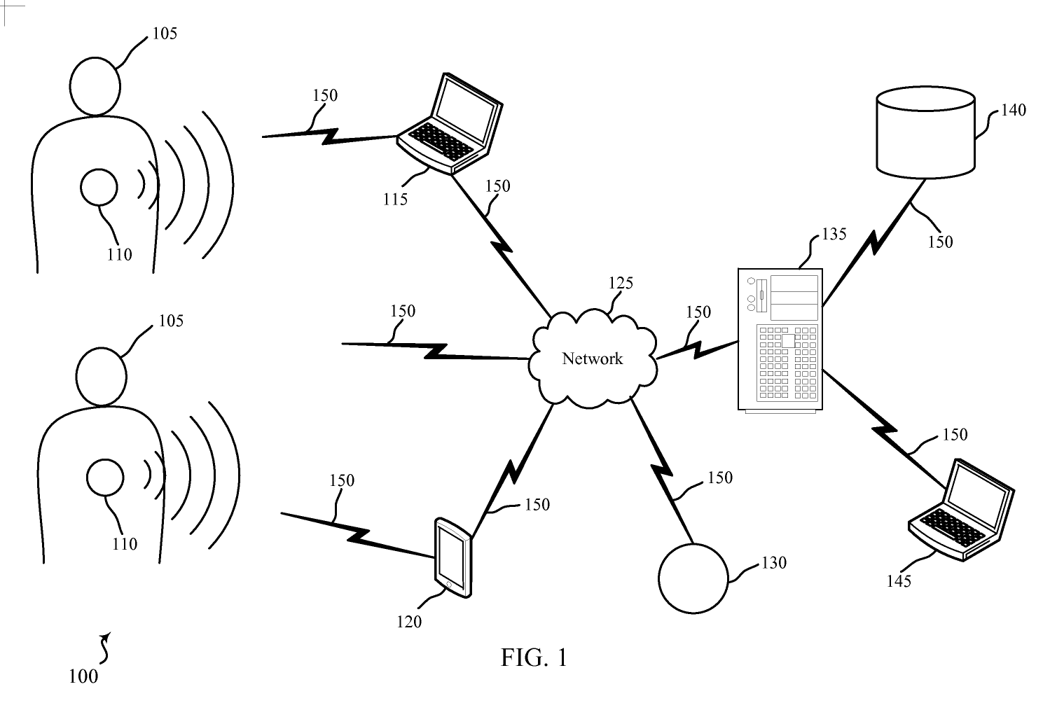

The disclosed wearable physiological sensing device may be a component of a physiological sensing system 100 of FIG. 1. The system 100 includes patients 105, each wearing a sensing device 110 that may be an example of the wearable physiological sensing devices generally described above. The sensing devices 110 may transmit signals via wireless communication links 150. The transmitted signals may be transmitted to local computing devices 115, 120. Local computing device 115 may be a local caregiver's station, for example. Local computing device 120 may be a mobile device, for example. The local computing devices 115, 120 may be in communication with a server 135 via network 125. The sensing devices 110 may also communicate directly with the server 135 via the network 125. Additional, secondary sensing devices 130 may also communicate directly with the server 135 via the network 125. The server 135 may be in further communication with a remote computing device 145, thus allowing a caregiver to remotely monitor the patients 105. The server 135 may also be in communication with various medical databases 140 where the collected data may be stored.

The sensing devices 110 are described in greater detail below. Each sensing device 110, however, may be capable of sensing multiple physiological parameters. Thus, the sensing devices 110 may each include multiple sensors to detect patient physiological parameters such as blood oxygen saturation, blood flow, pulse rate, electrocardiogram (ECG) data, respiratory rate, temperature, skin resistance, electromyography (EMG) data, fat content, position, posture, and/or other biomechanical data, such as shivering. For example, a first sensor in a sensing device 110 may be an optical sensor operable to detect a patient's blood oxygen saturation, blood flow, and pulse rate. A second sensor within a sensing device 110 may be operable to detect a second physiological parameter. For example, the second sensor may be an electrocardiogram (ECG) sensing module, a breathing rate sensing module, and/or any other suitable module for monitoring any suitable physiological parameter. The data collected by the sensing devices 110 may be wirelessly conveyed to either the local computing devices 115, 120 or to the remote computing device 145 (via the network 125 and server 135). Data transmission may occur via, for example, frequencies appropriate for a personal area network (such as Bluetooth, WiFi, cellular or IR communications) or near-field or local or wide area network frequencies such as radio frequencies specified by the IEEE 802.15.4 standard or medical body area network (MBAN) frequencies specifically allocated for medical devices. In some embodiments, the sensing devices 110 may also include a human-readable display or a local alert function, and may include an LED, a haptic motor, a buzzer, etc., that may serve as a local alert.

In some embodiments, a patient may wear more than one sensing device 110, which may each be capable of sensing one or more physiological parameters of the patient. For example, sensing device 110 may be attached to a patient's wrist and may include an optical sensor, while another sensing device 110 may be attached to the patient's chest or to some other portion of the patient's body and may allow for tissue measurements such as water content, fat content, lung function, heart function, etc. As an example, the additional sensing device 110 may be, for example, a physiological sensing patch coupled to the patient's chest and operable to detect electrical signals associated with the patient's cardiac function (e.g., ECG signals), respiration, and/or any other suitable physiological or biomechanical parameter. The one or more sensing devices 110 on a patient's body may be wirelessly linked to each other.

One or more sensing devices 110 may include one or more of the sensing modules illustrated and described in U.S. Publication No. 2011/0257542, filed Apr. 15, 2011; U.S. Publication No. 2012/0143019, filed Jun. 6, 2011; U.S. Publication No. 2009/0227856, filed Dec. 19, 2008; U.S. Publication No. 2009/0281394, filed Jun. 25, 2009; U.S. Publication No. 2013/0144130, filed Jan. 30, 2012; U.S. application Ser. No. 14/279,051, filed May 15, 2014; U.S. application Ser. No. 14/279,003, filed May 15, 2014; U.S. Pat. No. 8,400,302, issued Mar. 19, 2013; and U.S. Pat. No. 8,079,247, issued Dec. 20, 2011, each of which is commonly owned and is incorporated herein by reference in its entirety.

The local computing devices 115, 120 may enable the patient 105 and/or a local caregiver to monitor the collected physiological data. For example, the local computing devices 115, 120 may be operable to present data collected from sensing devices 110 in a human-readable format. For example, the received data may be outputted as a display on a computer or a mobile device. The local computing devices 115, 120 may include a processor that may be operable to present data received from the sensing devices 110 in a visual format. The local computing devices 115, 120 may also output data and/or alerts in an audible format using, for example, a speaker. In alternate embodiments, the physiological data may be displayed directly on the sensing device 110. For example, the sensing device 110 may comprise a visual display operable to display the patient's heart rate, respiratory rate, blood oxygen saturation, and/or any other suitable physiological information. In this way, the sensing device 110 may be used as a training aid and/or a medical monitoring device that conveniently provides the patient or caregiver with information regarding the patient's vital statistics.

The local computing devices 115, 120 may be custom computing entities configured to interact with the sensing devices 110. In some embodiments, the local computing devices 115, 120 and the sensing devices 110 may be portions of a single sensing unit operable to sense and display physiological parameters. In another embodiment, the local computing devices 115, 120 may be general purpose computing entities such as a personal computing device, such as a desktop computer, a laptop computer, a netbook, a tablet personal computer (PC), an iPod®, an iPad®, a smart phone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computing devices 115, 120 may include memory, a processor, an output, and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computing devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensing devices 110.

The processor of the local computing devices 115, 120 may be operated to control operation of the output of the local computing devices 115, 120. The output may be a television, a liquid crystal display (LCD) monitor, a cathode ray tube (CRT) monitor, speaker, tactile output device, and/or the like. In some embodiments, the output may be used as a local alert function, and may include an LED, a haptic motor, a buzzer, etc. In some embodiments, the output may be an integral component of the local computing devices 115, 120. Similarly stated, the output may be directly coupled to the processor. For example, the output may be the integral display of a tablet and/or smart phone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computing devices 115, 120 to the output.

At least one of the sensing devices 110 may be operable to transmit physiological data to the local computing devices 115, 120 and/or to the remote computing device 145 continuously, at scheduled intervals, when requested, and/or when certain conditions are satisfied (e.g., during an alarm condition).

The remote computing device 145 may be a computing entity operable to enable a remote user to monitor the output of the sensing devices 110. The remote computing device 145 may be functionally and/or structurally similar to the local computing devices 115, 120 and may be operable to receive and/or send signals to at least one of the sensing devices 110 via the network 125. The network 125 may be the Internet, an intranet, a personal area network, a local area network (LAN), a wide area network (WAN), a virtual network, a telecommunications network implemented as a wired network and/or wireless network, etc. The remote computing device 145 may receive and/or send signals over the network 125 via communication links 150.

The remote computing device 145 may be used by, for example, a healthcare professional to monitor the output of the sensing devices 110. The remote computing device 145 may receive an indication of physiological data when the sensors detect an alert condition, when the healthcare provider requests the information, at scheduled intervals, and/or at the request of the healthcare provider and/or the patient 105.

The server 135 may be configured to communicate with the sensing devices 110, the local computing devices 115, 120, secondary sensing devices 130, the remote computing device 145 and databases 140. The server 135 may perform additional processing on signals received from the sensing devices 110, local computing devices 115, 120 or secondary sensing devices 130, or may simply forward the received information to the remote computing device 145 and databases 140. The databases 140 may be examples of electronic health records (“EHRs”) and/or personal health records (“PHRs”), and may be provided by various service providers. The secondary sensing device 130 may be a sensor that is not attached to the patient 105 but that still provides data that may be useful in connection with the data provided by sensing devices 110.

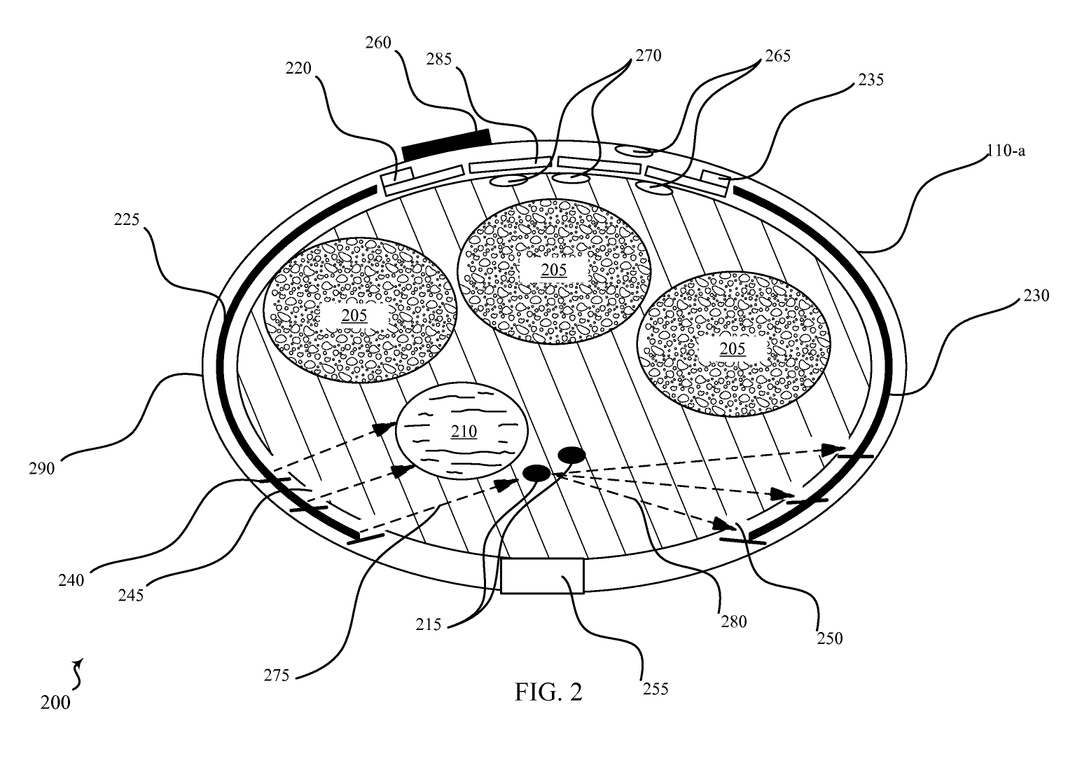

FIG. 2 includes a diagram 200 of an example wearable physiological sensing device 110-a, shown circumscribing a cross section of a patient's wrist. In diagram 200, sensing device 110-a may be an example of one or more sensing devices 110 illustrated in FIG. 1. The sensing device 110-a may include a band 290 or other securing means to couple the device to the patient's wrist or other extremity, as described in detail below.

The sensing device 110-a may include at least one light source 220, a first light pipe 225, a second light pipe 230, and an optical receiver 235. The at least one light source 220 may be operable to generate any one of an electromagnetic signal (or emit electromagnetic radiation), such as light (e.g., visible light, infrared light, ultraviolet light), and/or any other electromagnetic wave. The at least one light source 220 may be a light emitting diode, a laser, and/or any other suitable emitter. The at least one light source 220 may be a monochromatic emitter, a narrow band emitter, or a broad spectrum emitter. Because information regarding how efficiently light of various wavelengths travels through the human body may provide useful physiological data, the at least one light source 220 may be operable to emit light at multiple wavelengths. For example, because oxygenated blood and deoxygenated blood have different absorption characteristics in the red and near infrared wavelengths, the at least one light source 220 may be operable to emit red light and near infrared light.

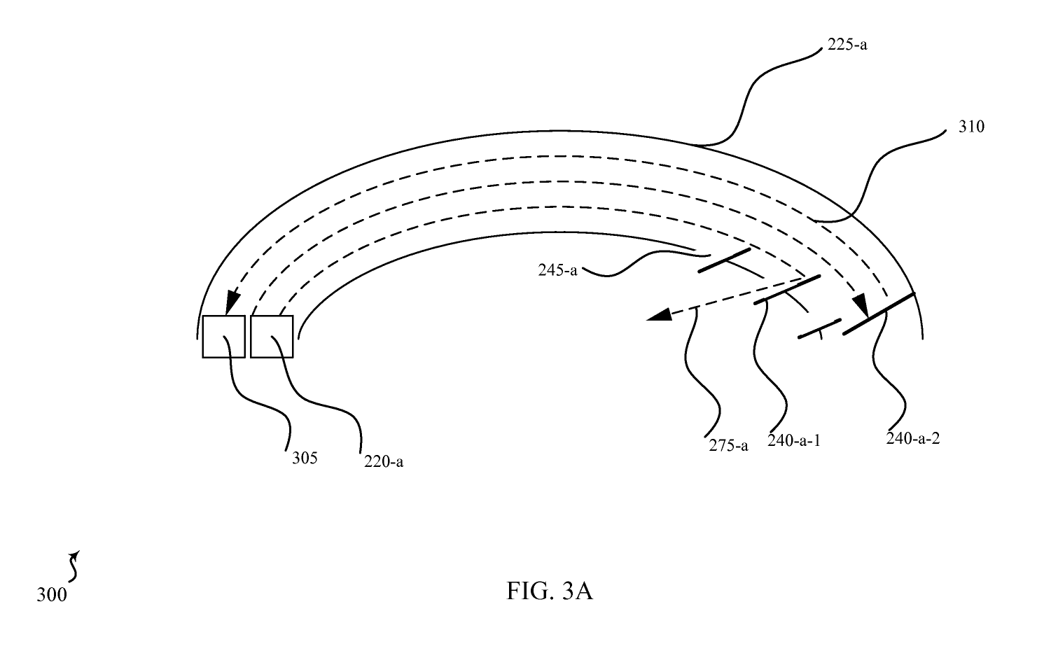

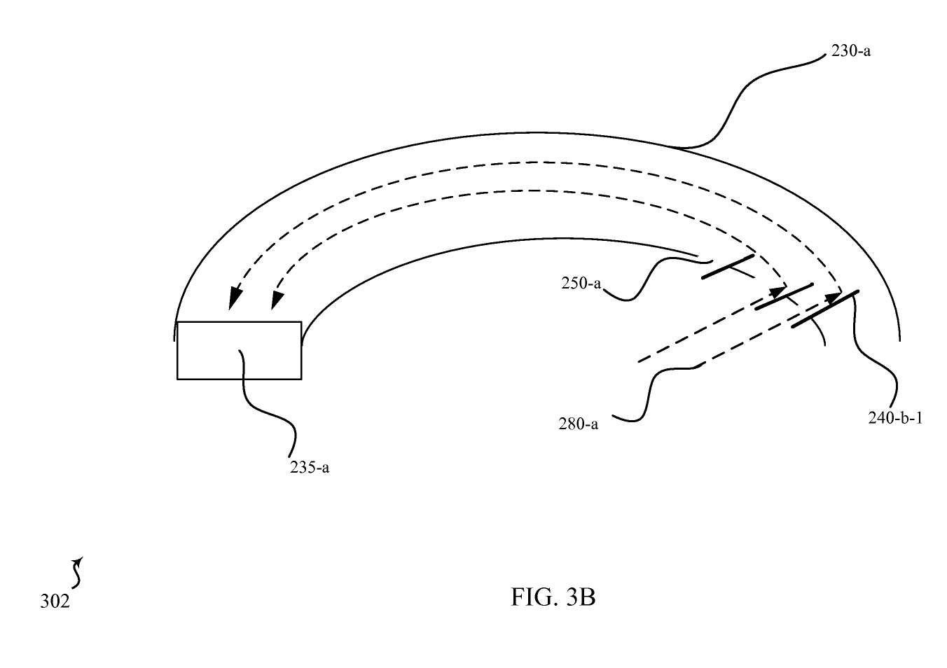

In some embodiments, the at least one light source 220 may comprise a waveform generator, operable to sweep, pulse, and/or otherwise modulate the light emitted by the at least one light source 220. For example, the waveform generator may cause the at least one light source 220 to alternately pulse beams of light having different wavelengths. In some embodiments, the waveform generator may allow the at least one light source 220 to operate more efficiently than, for example, a steady state or always-on light source by decreasing the duty cycle of the at least one light source 220. In some embodiments, the duty cycle of the at least one light source 220 may be adjusted to increase or decrease the resolution of the measured physiological parameters based on, for example, the activity level of the patient and/or the rate at which the measured physiological parameters change. In some embodiments, the at least one light source 220 may be ambient light. In one such embodiment, a reference optical receiver (described, for example, with reference to optical receiver 305 of FIG. 3) may pick up ambient light directly while a measuring optical receiver 235 may pick up the light that has radiated through the patient's extremity. The optical receiver 235 may, either individually or with a coupled processor 285, compare the two light measurements and may calculate physiological parameters, as described in greater detail below.

Each of first light pipe 225 and second light pipe 230 may at least partially circumscribe the extremity of the patient, in some embodiments extending from a posterior side to an anterior side of the patient's wrist. In some embodiments, the first light pipe 225 may comprise a plurality of first light pipes (not shown), such that light radiated from the at least one light source 220 may travel down one or more of the plurality of first light pipes, as described in further detail below. The first light pipe 225 and second light pipe 230 may comprise an optical fiber, an acrylic member, and/or any other suitable light transmitting material. The first light pipe 225 and second light pipe 230 may be coupled with or integral with the band 290 circumscribing the patient's extremity. The first light pipe 225 and second light pipe 230 may comprise at least one transmitting aperture 245 and at least one receiving aperture 250, respectively, spaced along the length of each light pipe. Each of first light pipe 225 and second light pipe 230 may further comprise a plurality of internal reflectors 240, which may be operable as diffusion lenses to “sparkle” or radiate light through the at least one transmitting aperture 245 into the extremity of the patient by one or more beams, or may alternatively be operable as a reflector to reflect light back up the first light pipe 225 (as shown in FIG. 3A), or to reflect light radiated into the extremity of the patient and received at the at least one receiving aperture 250 up the second light pipe 230 to the optical receiver 235 (as shown in FIG. 3B). In this way, although the at least one light source 220 and the optical receiver 235 are in some embodiments positioned at the posterior of the patient's wrist, light radiated from the at least one light source 220 may travel down the one or more first light pipes 225 to be radiated into the patient's wrist at a plurality of positions on the anterior side of the patient's wrist, and may be received at a plurality of positions on the anterior side of the patient's wrist before travelling up the second light pipe 230 to be received at the optical receiver 235. In some embodiments, either or both the first and second light pipes 225, 230 and the plurality of internal reflectors 240 may have optically selective properties, such as polarization or wavelength selectivity.

The optical receiver 235 may be any suitable photo detector, such as a photodiode, a photovoltaic device, a charge-coupled device, and/or any other suitable device to measure radiated light. In some embodiments, the sensing device 110-a may comprise a plurality of additional optical receivers (not shown) configured to receive light from the at least one light source 220. In some embodiments, each of the plurality of optical receivers may output a different signal representative of a different physiological parameter of the patient.

The sensing device 110-a may further comprise a plurality of physiological sensors, such as skin resistance sensors 270, temperature and heat flux sensors 265, and ECG touchpoint 260. The ECG touchpoint 260 may comprise a conductive portion operable to contact the skin of the patient in order to detect electrical signals associated with cardiac function. The ECG touchpoint 260 may send ECG data to a processor 285 and/or a memory (not shown) for processing and/or storage. The skin resistance sensors 270 may include two conductive portions operable to contact the skin of the patient, as well as an ohm meter (not shown). The skin resistance sensors 270 may be operable to detect perspiration and/or any other suitable conductivity related measurements, which may be transmitted to the processor 285. The temperature and heat flux sensors 265 may be operable to detect patient body temperature and/or heat flux. The temperature and heat flux sensors 265 may comprise one or more thermocouples. For example, a first thermocouple may be disposed adjacent to the skin of the patient's extremity, and the second thermocouple may be disposed on the outer surface of the sensing device 110-a. The first thermocouple may detect body temperature, and the second thermocouple may detect surface temperature of the sensing device 110-a, such that the difference between the two temperatures may be used to calculate heat flux. Data relating to the calculated heat flux may similarly be transmitted to the processor 285. In some embodiments, body temperature and/or heat flux may be used to calculate metabolic output of the patient.

A circuit board, battery, and/or processor 285 may be housed together with the physiological sensors on one portion of the sensing device 110-a, referred to in the aggregate as a sensor package. The at least one light source 220 and optical receiver 235 may also comprise the sensor package. In some embodiments, the sensor package may be positioned on the posterior of the patient's wrist. In other embodiments, one or more components of the sensor package may be distributed throughout the band of the sensing device 110-a.

In some embodiments, the first light pipe 225 and second light pipe 230 may comprise a band circumscribing an extremity of the patient, the extremity of the patient selected from any one of a wrist, ankle, thigh, waist, chest, neck, or ear, or any other suitable extremity. In some embodiments, at least one of the first light pipe 225 and second light pipe 230 may be a component of the band. Thus, the first light pipe 225 and second light pipe 230 may be surrounded or substantially surrounded by the band and/or the extremity of the patient, such that background light (i.e., light not generated by the at least one light source 220) entering either the first light pipe 225 and/or the second light pipe 230 may be eliminated or reduced. Further, incorporating the light pipes into the band may result in multiple light pipes having favorable optical coupling to the patient's body, which may reduce movement-related sensor errors. The band may be coupled to the patient using coupling mechanism 255, which may mechanically couple the first light pipe 225 and second light pipe 230 components of the band, such as by snaps, a buckle, hook and loop fasteners, and/or any other suitable electrical, mechanical and/or magnetic mechanism configured to secure the sensing device 110-a to the patient's extremity. In other embodiments, the band may be comprised of a sufficiently stiff or conformable material such that the sensing device 110-a may remain on the patient's extremity without the need for a coupling mechanism 255. In this way, the sensing device 110-a may be worn loosely around the patient's wrist or other extremity, similarly to a watch or a bracelet. In some embodiments, the sensor package and the band may be separate devices that are optically, electronically, and mechanically coupled together; in other embodiments, the sensor package and band may be formed as one continuous device. Thus, in some embodiments the sensor package may be coupled to the band and, similarly to a watch face, may be disposed on the posterior of the patient's wrist, with the first and second light pipes 225, 230 comprising the band extending to the anterior of the patient's wrist. Positioning the sensor package on the posterior of the wrist may provide a more comfortable fit for patients, as the bulk of the weight of the device will be positioned on top of the wrist.

In one embodiment (not shown), the at least one light source 220 and/or the optical receiver 235 may be disposed within the band on an anterior portion of the patient's wrist. In such an embodiment, in lieu of first light pipe 225 and second light pipe 230, the sensing device 110-a may comprise conductors disposed within the band operable to send power and/or signals from a battery located posterior to the wrist and/or send measurement data to a processor located posterior to the wrist.

In operation, light radiated from at least one light source 220 may travel down the first light pipe 225 or plurality of first light pipes, and may be reflected by the plurality of internal reflectors 240 through the at least one transmitting aperture 245 into the extremity of the patient. In other embodiments, as illustrated in FIG. 3A and discussed below, at least a portion of the light radiated from the at least one light source 220 may be reflected by the plurality of internal reflectors 240 back up the one or more first light pipes 225 to an optical receiver positioned adjacent to the at least one light source 220. In the embodiment illustrated in diagram 200, the patient's extremity viewed in cross section is a wrist. As the light travels out through the at least one transmitting aperture 245 through the patient's wrist, some of the light may come into contact with the patient's bone 210 or muscle 205, or other physiological “noise,” which may result in scattering of the light or loss of optical coupling such that the light is not properly received at the optical receiver 235. By providing at least one transmitting aperture 245 spaced along the length of the first light pipe 225, the sensing device 110-a is better able to ensure optical coupling, regardless of positioning of the sensing device 110-a on the patient's extremity. Thus, at least a portion of the light radiated into the extremity 275 may come into contact with the patient's vasculature 215, and may then be received at the at least one receiving aperture 250 of the second light pipe 230. Light received through the at least one receiving aperture 250 may be reflected by the plurality of internal reflectors 240 positioned in the second light pipe 230 up the second light pipe 230 to the optical receiver 235 positioned at the posterior of the patient's wrist. The optical receiver 235 may receive the light and may output one or more signals representative of the received light. The processor 285 may then determine one or more physiological parameters of the patient based, at least in part, on the one or more signals outputted from the optical receiver 235.

Light radiated from the at least one light source 220, through the extremity of the patient, and received at the optical receiver 235 may be measured by the optical receiver 235 to determine various physiological parameters of the patient. For example, the optical receiver 235, in conjunction with the processor 285, may process the relative attenuation of various wavelengths of the received light to determine physiological parameters. In one embodiment, because oxygenated blood has different absorption characteristics than deoxygenated blood, the optical receiver 235 may perform absorption spectrophotometry analysis to determine the blood oxygen saturation of the patient based on the relative attenuation of various wavelengths of light received.

As described above with respect to FIG. 1, the one or more physiological parameters of the patient may be displayed locally at the sensing device 110-a, or may be transmitted to any one or more of the local computing devices 115, 120 or remote computing device 145 for observation by the patient or caregiver.

FIG. 3A illustrates a diagram 300 of a first light pipe 225-a, which may be an example of the first light pipe 225 of FIG. 2. Diagram 300 illustrates an embodiment in which light radiated from at least one light source 220-a, which may be an example of the at least one light source 220 of FIG. 2, may travel down the length of the first light pipe 225-a. The light radiated from at least one light source 220-a may come into contact with a plurality of internal reflectors 240-a-1, 240-a-2 positioned along the length of the first light pipe 225-a. As illustrated in the embodiment shown by diagram 300, a portion of the radiated light coming into contact with internal reflector 240-a-2 will be reflected back up the first light pipe 225-a to come into contact with an optical receiver 305, which may or may not be the same optical receiver 235 illustrated in FIG. 2. As shown in diagram 300, another portion of the radiated light will come into contact with an optical reflector 240-a-1, which may reflect the radiated light out through at least one transmitting aperture 245-a, which may be an example of the at least one transmitting aperture 245 illustrated in FIG. 2, into the extremity of the patient. Although first light pipe 225-a is illustrated in diagram 300 as a single light pipe, in alternate embodiments first light pipe 225-a may comprise a plurality of first light pipes.

In some embodiments, first light pipe 225-a may not be constructed of an ideal optical material, such that first light pipe 225-a may absorb and/or scatter a portion of the light radiated through first light pipe 225-a from the at least one light source 220-a. Because optical losses caused by first light pipe 225-a may introduce noise and/or errors into optical-based calculations, it may be desirable to measure the losses, which may be used to apply a correction factor to improve the accuracy of an optical sensor. Thus, light radiated from the at least one light source 220-a that strikes internal reflector 240-a-2 may return along first light pipe 225-a to an optical receiver 305, which may detect light 310 that has traveled the length of the first light pipe 225-a without leaving first light pipe 225-a through the at least one aperture 245-a. The at least one light source 220-a may be operable to selectively direct light to internal reflector 240-a-2 such that the light 310 is reflected back to the optical receiver 305. In alternative embodiments, a first light source may direct light to internal reflector 240-a-2 in order to reflect light 310 back to the optical receiver 305, while a second light source (not shown) may direct light to a plurality of internal reflectors 240-a-1 to reflect light 275-a through the one or more apertures 245-a into the extremity of the patient. By measuring the light 310 reflected back to optical receiver 305, it may be determined that any losses in light that has reflected off the internal reflector 240-a-2 may be the result of optical defects in the first light pipe 225-a. The optical receiver 305 may therefore send a signal associated with the detected internal light to the processor 285, which may be operable to detect optical losses and/or apply a correction factor to optical measurements taken from light that passes through the vasculature of the wrist. In some embodiments, where first light pipe 225-a and second light pipe 230-a may be constructed of the same material, second light pipe 230-a may be assumed to have the same optical deficiencies of first light pipe 225-a. In other embodiments, second light pipe 230-a may similarly measure light reflected from an internal reflector which may return to an optical detector in second light pipe 230-a in order to measure optical losses in second light pipe 230-a.

In some embodiments, the optical receiver 305 positioned in the first light pipe 225-a may be used to detect optical defects in the first light pipe 225-a, while the optical receiver 235-a (as illustrated in FIG. 3B) positioned in the second light pipe 230-a may be used to detect light 275-a radiated through the extremity of the patient. Because some portion of the light radiated from the at least one light source 220-a may be lost due to optical defects in the first light pipe 225-a (which optical defects may be imputed to the second light pipe 230-a, assuming the two light pipes are constructed of the same material), and at least some portion of the light radiated from the at least one light source 220-a may be lost due to scattering in the patient's extremity, the portion of light which may reach the optical receiver 235-a may be markedly lessened, such that the optical receiver 235-a may require a higher optical sensitivity than that of the optical receiver 305.

FIG. 3B illustrates a diagram 302 of a second light pipe 230-a, which may be an example of second light pipe 230 illustrated in FIG. 2. Light 280-a radiated through the extremity of the patient may enter the second light pipe 230-a through the at least one receiving aperture 250-a, and may be reflected off the plurality of internal reflectors 240-b-1. The plurality of internal reflectors 240-b-1 may direct the light 280-a up the second light pipe 230-a to the optical receiver 235-a. The optical receiver 235-a may receive the light and may output one or more signals representative of the received light. The processor 285 (as shown in FIG. 2) may then determine one or more physiological parameters of the patient based, at least in part, on the one or more signals outputted from the optical receiver 235-a. As discussed above, in some embodiments, second light pipe 230-a may further comprise at least one light source and an internal reflector for reflecting light radiated from the at least one light source back up the second light pipe 230-a in order to detect optical losses in the second light pipe 230-a (not shown).

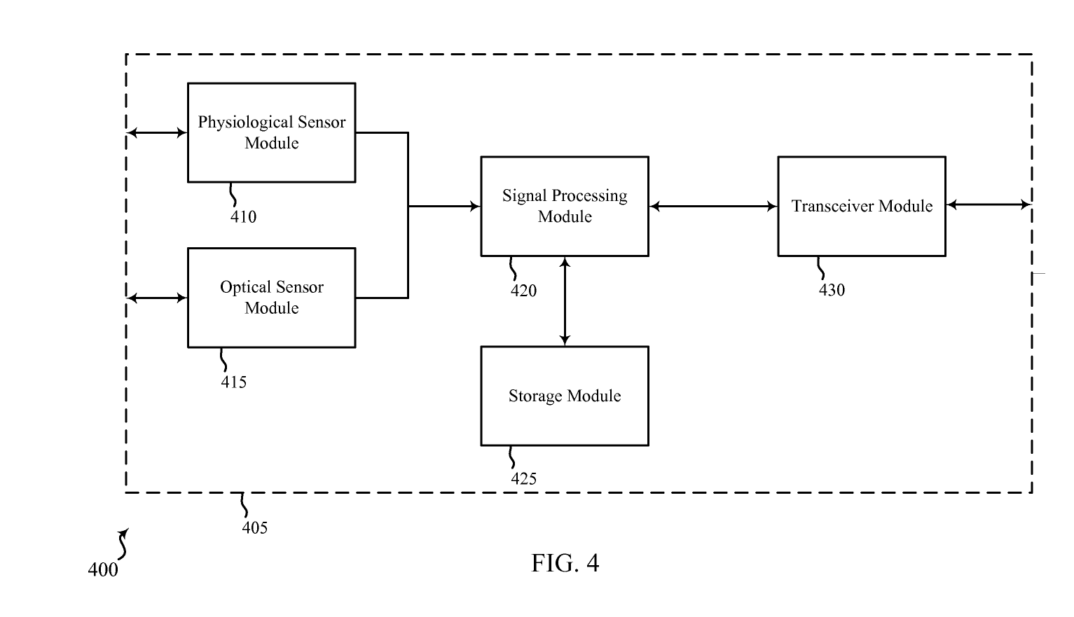

FIG. 4 is an example of a block diagram 400 of an apparatus 405 that may be used for sensing and reporting physiological parameters, in accordance with various aspects of the present disclosure. In some examples, the apparatus 405 may be an example of aspects of one or more of the sensing devices 110 described with reference to FIGS. 1, 2, and/or 3A-3B, and may sense and transmit physiological data. The apparatus 405 may also be a processor. The apparatus 405 may include a physiological sensor module 410, an optical sensor module 415, a signal processing module 420, a storage module 425, and/or a transceiver module 430. Each of these components may be in communication with each other.

The components of the apparatus 405 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

In some examples, the apparatus 405 may comprise a physiological sensor module 410 and an optical sensor module 415. In other embodiments, physiological sensor module 410 and optical sensor module 415 may comprise a single sensing module. For example, as described above with respect to FIG. 2, a physiological sensor module 410 may be operable to detect a first physiological parameter via a first sensing module, for example ECG touchpoint 260. Optical sensor module 415 may be operable to detect radiated light through the patient's extremity via either a second sensing module or the first sensing module. As additional examples, the physiological sensor module 410 may include an accelerometer operable to detect a patient's posture and/or activity level. Thus, the physiological sensor module 410 may be operable to determine whether the patient is standing, sitting, laying down, and/or engaged in physical activity, such as running. The physiological sensor module 410 may also be operable to detect a second physiological parameter. For example, the physiological sensor module 410 may further include a skin temperature sensing module, a breathing rate sensing module, and/or any other suitable module for monitoring any suitable physiological parameter.

In some examples, the signal processing module 420 includes circuitry, logic, hardware and/or software for processing the signals output by the physiological sensor module 410 and the optical sensor module 415. The signal processing module 420 may include filters, analog-to-digital converters and other digital signal processing units. In some embodiments, the signal processing module 420 may collect data from the one or more secondary sensing devices 130, optionally in conjunction with the data collected from the one or more sensing devices 110, to calculate derivative physiological data. For example, receiving pulse-timing data from multiple sensing devices 110 and/or secondary sensing devices 130 disposed on different parts of the patient's body may allow for the calculation of pulse transit time, blood pressure, and other time variant physiological parameters. Data processed by the signal processing module 420 may be stored in a buffer, for example, in the storage module 425. The storage module 425 may include magnetic, optical or solid-state memory options for storing data processed by the signal processing module 420.

In some examples, the transceiver module 430 may be operable to send and/or receive signals between the sensing devices 110 and either the local computing devices 115, 120 or the remote computing device 145 via the network 125 and server 135. In an embodiment, the transceiver module 430 may receive data from other sensing devices 110 or apparatuses 405 and may then transmit the data collected from multiple sensing devices 110 or apparatuses 405 to either the local computing devices 115, 120 or the remote computing device 145. The transceiver module 430 may include wired and/or wireless connectors. For example, in some embodiments, sensing devices 110 can be portions of a wired or wireless sensor network, coupled by the transceiver module 430. The transceiver module 430 may also be a wireless network interface controller (“NIC”), Bluetooth® controller, IR communication controller, ZigBee® controller and/or the like.

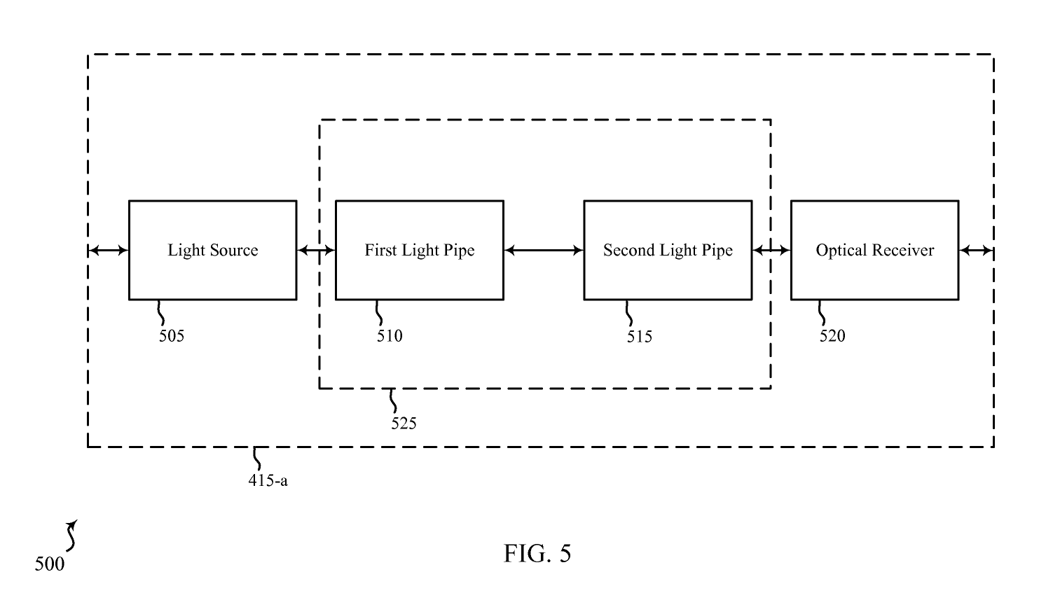

FIG. 5 is an example block diagram 500 of an optical sensor module 415-a that may be used for sensing and reporting optical data, in accordance with various aspects of the present disclosure. In some examples, the optical sensor module 415-a may be an example of aspects of the optical sensor module 415 described with reference to FIG. 4, and may further be an example of aspects of one or more of the sensing devices 110 described with reference to FIGS. 1, 2, and/or 3A-3B. The optical sensor module 415-a may sense and transmit optical data. Optical sensor module 415-a may comprise one or more light sources 505, a first light pipe 510, a second light pipe 515, and an optical receiver 520. In some embodiments, first light pipe 510 may comprise a plurality of first light pipes. First light pipe 510 and second light pipe 515 may be components of band 525, wherein the band 525 may at least partially circumscribe an extremity of a patient. As described above with reference to FIG. 2, light may be radiated at a single or plurality of wavelengths from one or more light sources 505, wherein one or more light sources 505 are optically coupled to first light pipe 510. Light radiated from one or more light sources 505 may travel through first light pipe 510, and may pass into the extremity of the patient. Light radiated into the extremity of the patient may then be received at the second light pipe 515, and may travel through second light pipe 515 to optical receiver 520, wherein optical receiver 520 may be optically coupled to second light pipe 515. Optical receiver 520 may then transmit a signal comprising optical data to signal processing module 420, as shown in FIG. 4.

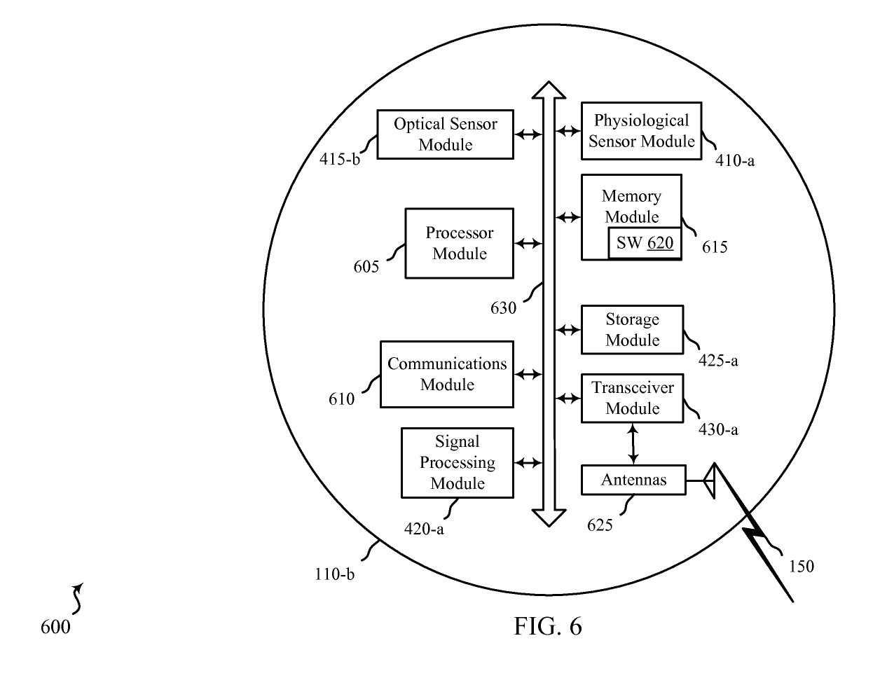

FIG. 6 shows a block diagram 600 of a sensing device 110-b for use in physiological sensing in accordance with various aspects of the present disclosure. The sensing device 110-b may have various configurations. The sensing device 110-b may, in some examples, have an internal power supply (not shown), such as a small battery, to facilitate mobile operation. Additionally, the sensing device 110-b may include a band (not shown) to circumscribe an extremity of a patient. Thus, in some examples, the sensing device 110-b may be an example of one or more aspects of one of the sensing devices 110 and/or apparatus 405 described with reference to FIGS. 1, 2, 4, and/or 5. The sensing device 110-b may be configured to implement at least some of the features and functions described with reference to FIGS. 1, 2, 3A, 3B, 4, and/or 5.

The sensing device 110-b may include an optical sensor module 415-b, a physiological sensor module 410-a, a processor module 605, a memory module 615, a communications module 610, at least one transceiver module 430-a, at least one antenna (represented by antennas 625), a storage module 425-a, or a signal processing module 420-a. Each of these components may be in communication with each other, directly or indirectly, over one or more buses 630. The optical sensor module 415-b, the physiological sensor module 410-a, the storage module 425-a, the transceiver module 430-a, and/or the signal processing module 420-a may be examples of the optical sensor module 415, the physiological sensor module 410, the storage module 425, the transceiver module 430, and/or the signal processing module 420, respectively, of FIG. 4.

The memory module 615 may include random access memory (RAM) or read-only memory (ROM). The memory module 615 may store computer-readable, computer-executable software (SW) code 620 containing instructions that are configured to, when executed, cause the processor module 605 to perform various functions described herein for communicating physiological data, for example. Alternatively, the software code 620 may not be directly executable by the processor module 605 but may be configured to cause the sensing device 110-b (e.g., when compiled and executed) to perform various of the functions described herein.

The processor module 605 may include an intelligent hardware device, e.g., a CPU, a microcontroller, an ASIC, etc. The processor module 605 may process information received through the transceiver module 430-a or information to be sent to the transceiver module 430-a for transmission through the antenna 625. The processor module 605 may handle, alone or in connection with the signal processing module 420-a, various aspects of signal processing.

The transceiver module 430-a may include a modem configured to modulate packets and provide the modulated packets to the antennas 625 for transmission, and to demodulate packets received from the antennas 625. The transceiver module 430-a may, in some examples, be implemented as one or more transmitter modules and one or more separate receiver modules. The transceiver module 430-a may be configured to communicate bi-directionally, via the antennas 625 and communication link 150, with, for example, local computing devices 115, 120 and/or the remote computing device 145 (via network 125 and server 135 of FIG. 1). Communications through the transceiver module 430-a may be coordinated, at least in part, by the communications module 610. While the sensing device 110-b may include a single antenna, there may be examples in which the sensing device 110-b may include multiple antennas 625.

As examples, the transceiver module 430-a may include a Bluetooth® module, an IEEE 802.15.4 module with custom stack, a ZigBee module, a wireless network interface controller (NIC), a cellular telephone module, and/or any other suitable module configured to send signals. The transceiver module 430-a may be operable to send a signal, for example over a network, the Internet, a cellular telephone link, and/or any other suitable communication means. In some embodiments, the transceiver module 430-a may include a short-range transmitter, for example, having a range of less than approximately 1000 feet.

The signal processing module 420-a may be used to interpret and process signals received from the optical sensor module 415-b and physiological sensor module 410-a. Using the received signals, the signal processing module 420-a may calculate physiological parameters, such as heart rate, respiratory rate, pulse rate, and so forth.

The sensing device 110-b may be operable to generate alerts, such as an audible alert, a visual alert, a haptic alert, and/or any other suitable type of alert. The sensing device 110-b may generate an alert when, for example, a module of the sensing device 110-b determines that a vital sign of a patient has exceeded a threshold. For example, the optical sensor module 415-b and physiological sensor module 410-a may detect a signal associated with a heart rate of the patient which the signal processing module 420-a may process and compare to a threshold such that if the patient's heart rate rises above a predetermined level and/or falls below a predetermined level, the sensing device 110-b may generate an alert.

The sensing device 110-b may include sensors such as accelerometers, gyroscopes, GPS modules, and so forth and may be operable to act as a pedometer, detect activity level, determine burned calories, and so forth. The collected data may be stored in the storage module 425-a, for example.

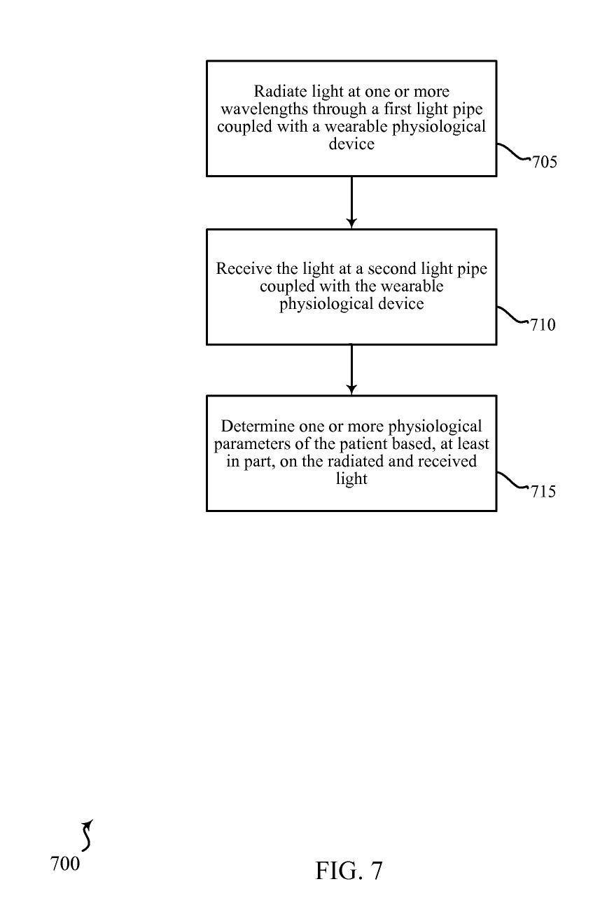

FIG. 7 is a flow chart illustrating an example of a method 700 for physiological sensing of a patient, in accordance with various aspects of the present disclosure. For clarity, the method 700 is described below with reference to aspects of one or more of the sensing devices 110 described with reference to FIGS. 1, 2, 4, 5 and/or 6, respectively, or aspects of one or more of the apparatus 405 described with reference to FIG. 4. In some examples, a sensing device such as one of the sensing devices 110 or an apparatus such as one of the apparatuses 405 may execute one or more sets of codes to control the functional elements of the sensing device or apparatus to perform the functions described below.

At block 705, the method 700 may include radiating light at one or more wavelengths through a first light pipe 225 coupled with a wearable physiological sensing device 110-a. As discussed above with reference to FIGS. 2 and 3A-3B, light radiated through the first light pipe 225 may be radiated into the extremity of the patient through at least one transmitting aperture 245 positioned along the length of the first light pipe 225. In addition, a plurality of internal reflectors 240 acting as lenses may transmit the light from the first light pipe 225 through the at least one transmitting aperture 245.

At block 710, the method 700 may include receiving the light at a second light pipe 230 coupled with the wearable physiological sensing device 110-a. As described above, the first and second light pipes 225, 230 may at least partially circumscribe the patient's extremity, and may comprise at least one aperture 245, 250 spaced along the lengths of the first and second light pipes 225, 230 such that light may be radiated into and received from a plurality of locations along the patient's extremity. As previously discussed, a portion of the light radiated into the extremity may come into contact with the patient's bones 210 or muscles 205, where the light may be scattered and may not complete an optical coupling with the optical receiver 235. Additionally, a portion of the light radiated into the extremity 275 may come into contact with the patient's vasculature 215, and may subsequently reach the at least one receiving aperture 250 positioned along the length of the second light pipe 230. Light received 280 through the at least one receiving aperture 250 may be reflected by a plurality of internal reflectors 240 positioned in the second light pipe 230, and may travel up the second light pipe 230 to the optical receiver 235.

At block 715, the method 700 may include determining one or more physiological parameters of the patient based, at least in part, on the radiated and received light. As previously described, the optical receiver 235 may receive the light radiated through the patient's extremity via the first and second light pipes 225, 230, and may output one or more signals representative of the received light. A processor 285 may receive the outputted one or more signals from the optical receiver 235 and may determine one or more physiological parameters of the patient based, at least in part, on the outputted signals. Such physiological parameters may comprise, for example, patient blood oxygen saturation or pulse rate.

In some embodiments, the wearable physiological sensing device 110-a may also receive input signals from one or more other sensing devices 110. The received input signals may be representative of received physiological data, and the processor 285 may receive the signals and determine, in some examples in conjunction with the signals outputted by the optical receiver 235, derivative patient physiological parameters. For example, the processor 285 may be operable to calculate the patient's pulse transit time based on signals received from the optical receiver 235 and one or more other sensing devices 110.

In some embodiments, the operations at blocks 705, 710 or 715 may be performed using the sensing devices 110 and/or apparatus 405 described with reference to FIGS. 1, 2, 4, 5, and/or 6. Nevertheless, it should be noted that the method 700 is just one implementation and that the operations of the method 700 may be rearranged or otherwise modified such that other implementations are possible.

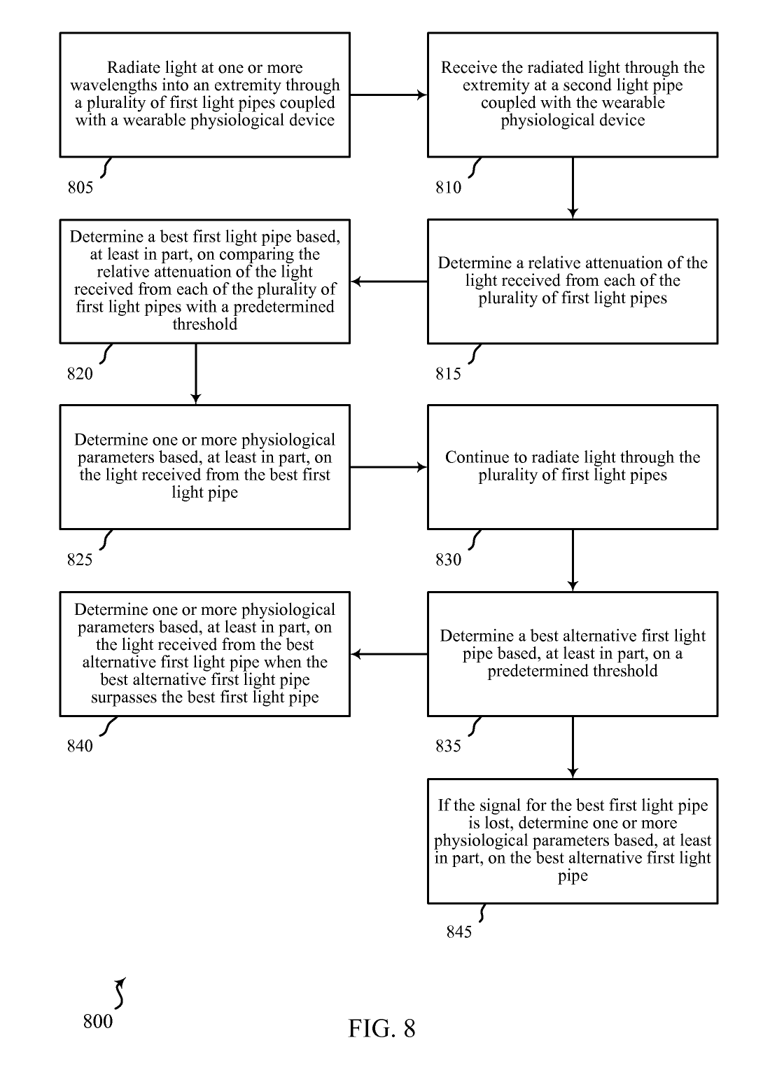

FIG. 8 is a flow chart illustrating an example of a method 800 for operating a search mode, in accordance with various aspects of the present disclosure. In some embodiments, a search mode may be used to determine the first light pipe having the best optical coupling at any given time, as well as a best alternative first light pipe, such that only one of a plurality of first light pipes need be used at any given time, and such that optical coupling may be maintained between the at least one light source and the optical receiver at any given time, despite patient and/or sensing device 110 movement. For clarity, the method 800 is described below with reference to aspects of one or more of the sensing devices 110 described with reference to FIGS. 1, 2, 4, 5 and/or 6, or aspects of one or more of the apparatus 405 described with reference to FIG. 4. In some examples, a sensing device such as one of the sensing devices 110 or an apparatus such as one of the apparatuses 405 may execute one or more sets of codes to control the functional elements of the sensing device or apparatus to perform the functions described below.

At block 805, method 800 may include radiating light at one or more wavelengths into an extremity of a patient through a plurality of first light pipes coupled with a wearable physiological sensing device. As previously discussed, a loosely fitting wearable physiological sensing device 110 may shift in orientation on a patient's wrist or other extremity during wearing, such that optical coupling between the one or more light source and the optical receiver may be lost, and physiological measurements may be interrupted or skewed. In order to maintain optical coupling and to ensure continuous and reliable monitoring of patient physiological measurements, light may be radiated through a plurality of first light pipes and into the extremity of the patient, such that at any given time and in any sensing device 110 orientation, at least one of the plurality of first light pipes may have optical coupling with the optical receiver through the patient's extremity via the one or more apertures positioned along the length of the plurality of first light pipes. Thus, light may be radiated at one or more wavelengths through a plurality of first light pipes during a search mode to determine the first light pipe(s) having optical coupling with the optical receiver at any given time.

At block 810, radiated light may be received through the extremity of the patient at a second light pipe coupled with the wearable physiological device. As previously discussed, at least one receiving aperture may be spaced along the length of the second light pipe in order to increase the probability that at least a portion of the light radiated into the extremity will reach the optical receiver. In some embodiments, the sensing device 110 may also or alternatively comprise a plurality of second light pipes in order to ensure optical coupling.

In order to determine the first light pipe(s) having optical coupling with the optical receiver at any given time, at block 815, the method 800 may include determining a relative attenuation of the light received from each of the plurality of first light pipes, and at block 820, the method 800 may include determining a best first light pipe based, at least in part, on comparing the relative attenuation of the light received from each of the plurality of first light pipes with a predetermined threshold. Based on the relative attenuation of the light received at the optical receiver, it may be determined which first light pipe(s) of the plurality of first light pipes has optical coupling with the optical receiver, and of those first light pipe(s) having optical coupling, which first light pipe is producing the strongest signal. The relative attenuation may be compared with a predetermined threshold in order to ensure that the light received is producing a sufficiently strong signal such that the optical receiver, in conjunction with the processor, may determine one or more physiological parameters of the patient.

Once it has been determined which first light pipe is producing the strongest signal (“the best first light pipe”), at block 825, method 800 may include determining one or more physiological parameters based, at least in part, on the light received from the best first light pipe.

Because the patient may continue to move, and therefore the wearable physiological sensing device 110 may move with the patient, optical coupling may be gained and lost on a continuous basis. Thus, even after a best first light pipe has been determined, light may continue to be radiated through the plurality of first light pipes at block 830 of method 800, in order to determine a best alternative first light pipe based, at least in part, on a predetermined threshold, as shown at block 835. By determining a best alternative first light pipe, should the optical coupling or signal for the best first light pipe be lost at any time, the one or more physiological parameters of the patient may be determined based, at least in part, on the best alternative first light pipe, as shown at block 845. Alternatively, should the signal for the best alternative first light pipe at any time surpass the signal for the best first light pipe, the one or more physiological parameters of the patient may similarly be determine based, at least in part, on the best alternative first light pipe, as shown at block 840. In this way, the wearable physiological sensing device 110 may provide for constant reliable patient physiological monitoring, regardless of any shifting of the sensing device 110 on the patient's extremity during normal wear and use.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that can be accessed by a general purpose or special purpose computer. By way of example, and not limitation, computer-readable medium can comprise RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and blu-ray disc where disks usually reproduce data magnetically, while discs reproduce data optically with lasers. Combinations of the above are also included within the scope of computer-readable media.

The previous description of the disclosure is provided to enable a patient skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A wearable physiological sensing device, comprising:
at least one light source;
a first light pipe coupled with the at least one light source, the first light pipe at least partially circumscribing an extremity of a patient, and comprising at least one aperture for radiating light from the at least one light source into the extremity;
a second light pipe comprising at least one aperture for receiving the light radiated through the extremity, the second light pipe at least partially circumscribing the extremity of the patient;
an optical receiver coupled with the second light pipe configured to receive the light and output one or more signals representative of the received light; and
a processor configured to determine one or more physiological parameters of the patient based, at least in part, on the outputted one or more signals.

2. The wearable physiological sensing device of claim 1, wherein the first light pipe comprises a plurality of first light pipes.

3. The wearable physiological sensing device of claim 2, wherein the processor is further configured to select a best first light pipe of the plurality of first light pipes based, at least in part, on comparing the one or more signals representative of the received light with a predetermined threshold.

4. The wearable physiological sensing device of claim 1, wherein the at least one aperture of the first light pipe comprises a plurality of apertures spaced along a length of the first light pipe and the at least one aperture of the second light pipe comprises a plurality of apertures spaced along a length of the second light pipe.

5. The wearable physiological sensing device of claim 1, further comprising:
a plurality of internal reflectors positioned in at least one of the first light pipe and the second light pipe.

6. The wearable physiological sensing device of claim 1, wherein the sensing device comprises a band positioned on the extremity of the patient, the extremity of the patient selected from any one of a wrist, ankle, chest, waist, neck, thigh, or ear.

7. The wearable physiological sensing device of claim 6, wherein at least one of the first light pipe and the second light pipe is a component of the band.

8. The wearable physiological sensing device of claim 1, wherein the at least one light source comprises a waveform generator, wherein the waveform generator is configured to modulate the light radiated from the at least one light source.

9. The wearable physiological sensing device of claim 1, further comprising a plurality of additional optical receivers configured to receive the light from the at least one light source.

10. The wearable physiological sensing device of claim 9, wherein each of the plurality of additional optical receivers outputs a different signal representative of a different physiological parameter of the patient.

11. The wearable physiological sensing device of claim 1, wherein the determined one or more physiological parameters comprise any one of blood oxygen saturation, blood flow, blood pressure, pulse transit time, pulse rate, respiratory rate, body temperature, heat flux, skin resistance, electrocardiogram (ECG) data, electromyography (EMG) data, position, posture, fat content, perspiration, or shivering, or combinations thereof.

12. A method of measuring physiological parameters, comprising:
radiating light at one or more wavelengths through a first light pipe coupled with a wearable physiological sensing device, the first light pipe at least partially circumscribing an extremity of a patient and having one or more apertures through which the radiated light is radiated into the extremity;
receiving the light at a second light pipe coupled with the wearable physiological sensing device, the second light pipe at least partially circumscribing the extremity and having one or more apertures through which the light is received from the extremity; and
determining one or more physiological parameters of the patient based, at least in part, on the radiated and received light.

13. The method of claim 12, further comprising:
outputting one or more signals associated with the one or more physiological parameters.

14. The method of claim 12, further comprising:
reflecting the light radiated through the first light pipe via a plurality of internal reflectors positioned in the first light pipe and/or reflecting light received through the extremity into the second light pipe via a plurality of internal reflectors positioned in the second light pipe.

15. The method of claim 12, further comprising:
receiving one or more signals at the physiological sensing device from one or more secondary physiological sensing devices positioned on one or more other locations on the body of the patient.

16. The method of claim 15, further comprising:
calculating one or more derivative physiological parameters of the patient based, at least in part, on at least one of the radiated and received light, or the one or more signals received from the one or more secondary physiological sensing devices.

17. The method of claim 12, wherein each of the one or more physiological parameters is associated with the light radiated from a different one of the one or more apertures of the first light pipe.

18. The method of claim 12, further comprising operating a search mode, wherein the search mode comprises:
radiating light into the extremity through a plurality of first light pipes;
receiving the radiated light through the extremity via the second light pipe;
determining a relative attenuation of the light received from each of the plurality of first light pipes;
determining a best first light pipe based, at least in part, on comparing the relative attenuation of the light received from each of the plurality of first light pipes with a predetermined threshold;
determining one or more physiological parameters based, at least in part, on the light received from the best first light pipe;
continuing to radiate light through the plurality of first light pipes;
determining a best alternative first light pipe based, at least in part, on the predetermined threshold; and
determining one or more physiological parameters based, at least in part, on the light received from the best alternative first light pipe when the best alternative first light pipe surpasses the best first light pipe.

19. The method of claim 18, wherein if a signal for the best first light pipe is lost, one or more physiological parameters are determined based, at least in part, on the best alternative first light pipe.

20. An apparatus for measuring physiological parameters, comprising:
means for radiating light at one or more wavelengths through a first light pipe coupled with a wearable physiological sensing device, the first light pipe at least partially circumscribing an extremity of a patient and having one or more apertures through which the radiated light is radiated into the extremity;
means for receiving the light at a second light pipe coupled with the wearable physiological sensing device, the second light pipe at least partially circumscribing the extremity and having one or more apertures through which the light is received from the extremity; and
means for determining one or more physiological parameters of the patient based, at least in part, on the radiated and received light.
